(19) United States
(12) Patent Application Publication
(10) Pub. No.: US 2026/0009753 A1
(43) Pub. Date: Jan. 8, 2026

(54) SYSTEM AND METHOD FOR DETERMINING A GAS CONCENTRATION USING A SENSOR DEVICE

(71) Applicant: Infineon Technologies AG, Neubiberg (DE)

(72) Inventor: Michael KANDLER, Sauerlach (DE)

(21) Appl. No.: 19/252,546

(22) Filed: Jun. 27, 2025

(30) Foreign Application Priority Data
Jul. 4, 2024 (DE) 102024206316.9

(51) Int. Cl. G01N 25/18; G01N 33/00

(52) U.S. Cl. CPC G01N 25/18; G01N 33/0062

(57) ABSTRACT

A sensor device for measuring a gas concentration includes a sensor element including a cavity having an opening for receiving a gas, a first measuring component and a first heating component arranged in the cavity in each case. The sensor element further includes a control circuit arrangement which is electrically coupled with the sensor element and set up to control operation of the sensor element, wherein the control circuit arrangement is set up to heat the first heating component by applying an electric heating voltage during a heating phase, to measure temperature changes at the first measuring component as a function of the heating during the heating phase, to ascertain a heat characteristic of the gas from the temperature changes, and to determine a concentration of a component of the gas from the heat characteristic that is ascertained.

### CROSS-REFERENCE TO RELATED APPLICATION

This application claims priority to Germany Patent Application No. 102024206316.9 filed on Jul. 4, 2024, the content of which is incorporated by reference herein in its entirety.

### TECHNICAL FIELD

The present disclosure relates to a sensor device for measuring a gas concentration and a method for determining a gas concentration, in particular concentrations of a plurality of gas components.

### BACKGROUND

Gas concentration sensors, e.g., thermal conductivity sensors, can be used for example in the automotive sector or in a wide range of industrial applications. Here, such sensors can provide measured values which indicate a thermal conductivity of a gas for analysis, from which in turn a concentration of a component of the gas, e.g., a hydrogen content, can be determined. However, these measured values may be affected by deviations or offset effects which are influenced based on other properties of the gas to be measured. For example, the measurement of thermal conductivity can be highly dependent on the ambient pressure of the gas to be measured. Furthermore, conventional thermal conductivity sensors are limited to determining a concentration of a single gas component. To determine a plurality of components of a gas and their concentrations, either additional sensor units or alternative sensor types are therefore required. For various applications, it may be desirable to offer a thermal conductivity sensor that provides reliable and accurate measurement results. Furthermore, it may be desirable to offer a thermal conductivity sensor that can determine the concentrations of different gas components. Furthermore, it may be desirable to provide suitable methods for the operation of such thermal conductivity sensors.

### SUMMARY

There is a need for a gas sensor device using which a gas concentration, in particular a hydrogen content, can be determined with higher operational reliability.

According to the present disclosure, these objects are achieved by the features of independent claim 1. Furthermore, further advantageous implementations emerge from the dependent claims and the description.

A first aspect of the present disclosure relates to a sensor device for measuring a gas concentration, which includes a sensor element having a cavity, an opening of the cavity for receiving a gas, and a first measuring component and a first heating component arranged in the cavity in each case. The sensor device further includes a control circuit arrangement which is electrically coupled with the sensor element and is set up to control operation of the sensor element. The control circuit arrangement is set up to heat the first heating component by applying an electric heating voltage during a heating phase, and to measure temperature changes at the first measuring component as a function of the heating during the heating phase. The control circuit arrangement is further set up to ascertain a heat characteristic of the gas from the temperature changes and to determine a concentration of a component of the gas from the heat characteristic that is ascertained.

A second aspect of the present disclosure relates to a method for determining a gas concentration. The method includes surrounding a first measuring component and a first heating component of a sensor element with a gas, heating the first heating component by applying an electric heating voltage during a heating phase, and measuring temperature changes at the first measuring component as a function of the heating during the heating phase. The method further includes ascertaining a heat characteristic of the gas from the temperature changes, and determining a concentration of a component of the gas from the heat characteristic that is ascertained.

A person skilled in the art will discern further features and advantages of the implementation upon reading the following detailed description and examining the attached drawings.

### BRIEF DESCRIPTION OF THE DRAWINGS

The present disclosure is shown in an example and non-limiting manner in the illustrations of the attached drawings, in which identical reference numbers refer to similar or identical elements. The elements in the drawings are not necessarily depicted to scale in relation to each other. The features of the various examples shown can be combined, provided that they are not mutually exclusive.

FIG. 1 shows a schematic illustration of a sensor device for measuring a gas concentration to illustrate the functional principle of thermal conductivity sensors.

FIG. 2A shows a schematic plan view of a first example implementation of a sensor element for use in a sensor device for measuring a gas concentration.

FIG. 2B shows a schematic cross section of the first example implementation of a sensor element.

FIG. 2C shows a schematic example circuit diagram of a control circuit arrangement for operating a sensor element, e.g., the first example implementation.

FIG. 3A shows a schematic plan view of a second example implementation of a sensor element for use in a sensor device for measuring a gas concentration.

FIG. 3B shows a schematic cross section of the second example implementation of a sensor element.

FIG. 3C shows a circuit diagram of a bridge circuit which may be contained in a sensor element according to the disclosure.

FIG. 4A shows a schematic plan view of a third example implementation of a sensor element for use in a sensor device for measuring a gas concentration.

FIG. 4B shows a schematic cross section of the third example implementation of a sensor element.

FIG. 4C shows a circuit diagram of a bridge circuit which may be contained in a sensor element according to the disclosure.

FIG. 5 shows a graph for illustrating the functional principle of the sensor device according to at least one of the example implementations and the typically clear temperature dependence of the measured values during the heating phase.

FIG. 6 shows a flowchart of a method for determining a gas concentration using a sensor device according to the disclosure.

### DETAILED DESCRIPTION

The implementations described below describe thermal conductivity sensors (or thermal conductivity gas sensors) and methods for operating such sensors according to this disclosure in detail. Thermal conductivity sensors as described here can be used in particular as gas sensors for the detection of hydrogen and/or hydrogen concentrations. Hydrogen sensors can be used in a wide range of applications, such as e.g., in the automotive field or in industrial applications. For example, hydrogen sensors can be used for detecting hydrogen exhaust gases, for monitoring exhaust gases, for monitoring batteries, for detecting hydrogen leaks, for detecting hydrogen in industrial plants, etc.

With regard to climate targets, the automotive industry is promoting and developing the production of hydrogen-powered vehicles. Fuel cell cars can be seen as a breakthrough for electromobility and can contribute considerably to reducing CO2 emissions. The thermal conductivity sensors described here improve the hydrogen technology and can thus contribute at least to some extent to achieving the set climate targets. The thermal conductivity sensors described here offer a simple and efficient option for detecting components of a gas that is to be measured. By comparison, the production and design of conventional sensors can be more complex and require a higher number of components, which leads to increased resource consumption. The thermal conductivity sensors described here save resources and can contribute to energy savings. Overall, improved thermal conductivity sensors according to the disclosure and methods for operating such sensors can contribute to green technology and green energy solutions, e.g., to climate-friendly solutions with reduced energy consumption.

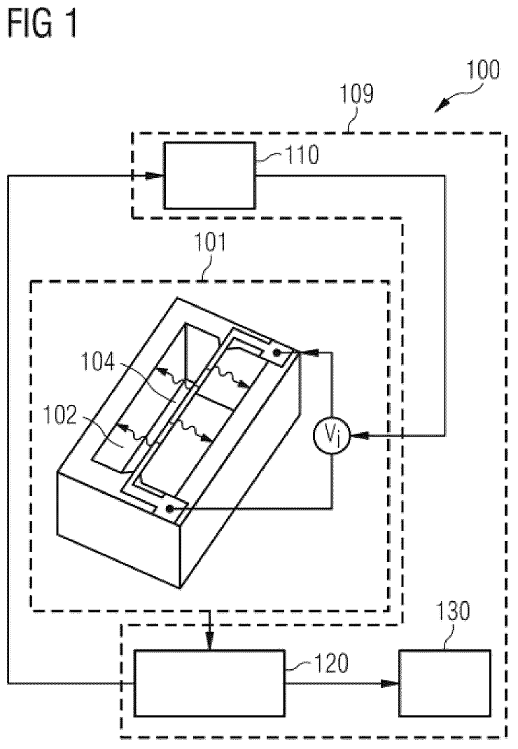

FIG. 1 illustrates the functional principle of a thermal conductivity sensor and shows a sensor device 100 for measuring a gas concentration. The sensor device 100 comprises a sensor element 101 which can be a thermal conductivity sensor. The sensor element 101 comprises a measuring component 104 which can be configured as a freely suspended bar or wire resistance element. The measuring component 104 can be produced for example based on MEMS technology (microelectromechanical systems). The measuring component 104 is arranged in a cavity 102 in which it is exposed to the gas to be measured. In other words, a gas for analysis can surround the measuring component 104 in the cavity 102. The gas to be measured can be introduced into the cavity 102 through an opening 103 (not shown in the figure for reasons of clarity). If a supply voltage Vⁱ is applied to the measuring component 104 as illustrated, the measuring component 104 can act as a heating wire and heat up to a stable characteristic temperature Tᵢ above the ambient temperature. The measuring component 104 can release thermal energy to the surrounding gas, which is illustrated in FIG. 1 by small arrows pointing away from the measuring component 104. When the stable characteristic temperature Tᵢ is reached, the total heat loss of the measuring component 104 may correspond to the energy generated by the supply voltage Vᵢ. The higher the thermal conductivity of the gas in the cavity 102, the greater its cooling effect and the lower the temperature of the measuring component 104 at a constant supply voltage Vᵢ. In one example, the measuring component 104 can be a positive temperature coefficient (PTC) resistor which is configured such that it conducts electrical currents better at low temperatures than at high temperatures. This means that the resistance value of the measuring component 104 may be lower at low temperatures than at high temperatures. Consequently, the electric current through the measuring component 104 can be a measure for the thermal conductivity of the surrounding gas.

The sensor device 100 further comprises a control circuit arrangement 109 with a voltage supply 110, a control unit 120 and an evaluation unit 130. The voltage supply 110 is set up to apply the supply voltage Vᵢ to the measuring component 104. The voltage supply 110 can output e.g., various supply voltages Vᵢ. The control circuit arrangement 109 further comprises a control unit 120 which is electronically coupled with the voltage supply 110. The control unit 120, for example a control circuit, can be set up to supply the voltage supply 110 with a trigger signal, based on which the voltage supply 110 applies a supply voltage Vᵢ to the measuring component 104. The control unit 120 is further set up to detect a sensor value from the sensor element 101. For example, the control unit 120 detects a set of output values Oᵢ of the sensor element 101 when the supply voltage Vᵢ is applied at certain predefined measurement times tᵢ during a heating phase. The predefined measurement times tᵢ can be defined in such a way that an output signal Oᵢ is detected at regular intervals after the supply voltage Vᵢ has been applied. The output signals Oᵢ can e.g., be an electric current through the measuring component 104.

The control circuit arrangement 109 further comprises an evaluation unit 130, for example an evaluation circuit, which is electronically coupled with the control unit 120 and set up to receive the output signals Oᵢ from the control unit 120 and to determine a concentration of a component of a gas in the cavity 102 of the sensor element 101 from them. For example, the evaluation unit 130 is set up to determine temperature changes at the first measuring component 104 from the output signals Oᵢ as a function of the heating (e.g., a function of heating of a heating component). Furthermore, the evaluation unit 130 is set up to ascertain a heat characteristic of the gas in the cavity 102 from the temperature changes and to determine a concentration of a component of the gas from the heat characteristic, e.g., a heating curve. The evaluation unit 130 can further be set up to output the ascertained concentration of the gas component or a signal based on it.

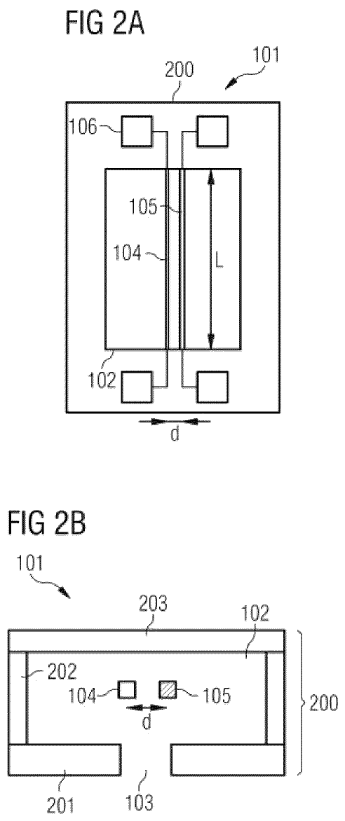

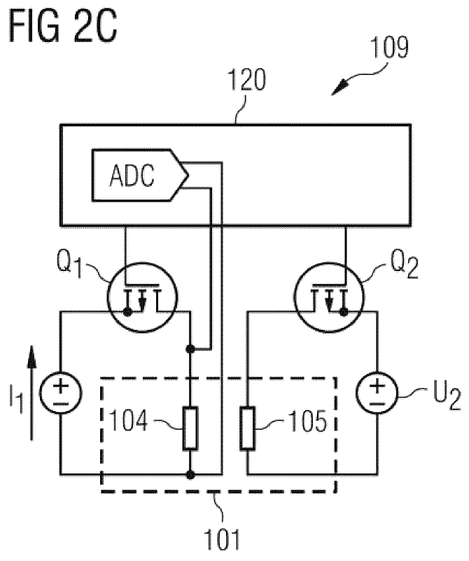

FIG. 2A shows a schematic plan view of a first example implementation of a sensor element 101 for use in a sensor device 100 for measuring a gas concentration. In contrast to conventional sensor elements 101, the first example implementation is based on the separation of measuring component 104 and heating component 105. The sensor element 101 comprises a cavity 102 which is formed for example by a recess in a housing 200. The sensor element 101 further comprises a measuring component 104 and a heating component 105 that is separate from the measuring component 104. The measuring component 104 and the heating component 105 are each formed by a bridge that is anchored in the substrate 200, which bridges are electrically coupled to contact surfaces 106 to form an electrical connection to the control circuit arrangement 109, e.g., by wire bonding. The bridges of the measuring component 104 and the heating component 105 have a length L which corresponds for example to a length of the cavity 102. The measuring component 104 and the heating component 105 are arranged at a distance d from one another. For example, the distance d is less than the length L of the bridges, but greater than a mean free path length of the gas or a component of the gas which is located in the cavity 102 and surrounds the measuring component 104 and the heating component 105.

FIG. 2B shows a schematic cross section of the first example implementation of a sensor element 101. The sensor element 101 can be a semiconductor component which comprises a housing 200 which is formed from a substrate 201, sidewall elements 202 and a cover 203. The housing 200 thus forms a boundary of the cavity 102 and defines same. In this example, the substrate 201 comprises the opening 103 to surroundings of the sensor element 101, so that gas for analysis that is to be measured can enter or be introduced into the cavity 102. The opening 103 can alternatively also be arranged in a sidewall element 202 or in the cover 203. The measuring component 104 and the heating component 105 are arranged in the cavity 102, so both can come into contact with the gas in the cavity. For example, the measuring component 104 and the heating component 105 are each formed by a MEMS wire which forms a piezoresistive element. The measuring component 104 and the heating component 105 are formed from an electrically conductive material. Via contact surfaces 106 (cf. FIG. 2A), it is possible to apply a heating voltage Vₕ to the heating component 105 and read out the measuring component 104. For example, a resistance value of the measuring component 104 can be read out via the contact surfaces 106. Alternatively, it is possible, via the contact surfaces 106, to apply a measuring voltage Vᵢₙ to the measuring component 104, via which a measured value Vₒᵤₜ can then be ascertained.

FIG. 2C shows a schematic example circuit diagram of a control circuit arrangement 109 for operating a sensor element 101, e.g., the first example implementation according to FIGS. 2A and 2B. The heating component 105 that is illustrated by a resistor is heated up, e.g., periodically using heating pulses, using an electric voltage Vₕ which is generated by a control unit 120, a voltage source U₂ and a switching element Q₂, e.g., a transistor, and applied to the heating component 105. A temperature of the measuring component 104, likewise illustrated in FIG. 2C by a resistor, is measured e.g., periodically using a circuit which consists of a current source I₁, a further switching element Q₁, e.g., likewise a transistor, and an analog-to-digital converter ADC. For measuring temperature changes, the control circuit arrangement 109 is set up to measure a temperature of the measuring component 104, or a temperature-dependent measurement variable such as e.g., a resistance, during the heating phase by applying an electric measuring voltage Vᵢₙ at predefined measurement times.

Owing to the presence of the gas to be analyzed in the cavity 102, the measuring component 104 is also heated while the heating voltage Vₕ is applied at the heating component 105. A temperature of the measuring component 104 depends on the distance d, on the heating voltage Vₕ, and on the thermal conductivity and the heat capacity of the gas in the cavity 102. Since the distance d is fixed and the heating voltage Vₕ can be applied in a controlled manner, for example by a constant voltage or by voltage pulses of predetermined form and period, a heat-up time and a maximum temperature of the measuring component 104 during the heating phase therefore essentially only depend on the thermal conductivity and the heat capacity of the gas in the cavity 102. If the temperature or a temperature-dependent measured value, e.g., a resistance, of the measuring component 104 is detected at regular time intervals during the heating phase, a heat characteristic, e.g., a heating curve, can be ascertained from these temperature changes, the gradient or heat-up time of which depends on the heat capacity and the maximum temperature of which depends on the thermal conductivity of the gas in the cavity 102. An impedance of the measuring component 104 can be significantly greater than the impedance of the heating component 105. This efficiently avoids self-heating of the measuring component 104 at an applied measuring voltage Vᵢₙ. Furthermore, the control circuit arrangement 109 can be set up to read out the measuring component 104 only selectively at regular intervals during the heating phase, which likewise avoids self-heating. A low impedance of the heating component 105 by contrast allows an effective and rapid heating up of the same.

An evaluation unit 130 can ascertain the heat characteristic from the measured values and determine a heating curve with gradient and maximum value, e.g., a maximum temperature, for example from that. From the heat characteristic, the evaluation unit 130 can then determine the thermal conductivity and heat capacity of the gas in the cavity 102. The evaluation unit 130 may further comprise a memory in which reference values for different gases and/or gas compositions are stored. The thermal conductivity and heat capacity that are ascertained can be compared with the reference values to determine a concentration of a component of the gas. For example, a hydrogen concentration in air can be ascertained. Alternatively, the evaluation unit 130 can be set up to compare a gradient and a maximum value of the heating curve directly with reference values in order to determine the concentration of the gas component. For example, the reference values in this case are calibrated gradients and maximum values for known gas concentrations in air in the relevant concentration range.

Furthermore, the control circuit arrangement 109, or an evaluation unit 130 of the control circuit arrangement 109, can be set up to determine a first concentration of a first component of the gas and a second concentration of a second component of the gas from the heat characteristic that is ascertained. In the case of gas components that are known in principle, e.g., H₂ and H₂O in air, the evaluation unit 130 can be set up to determine the concentration of these two components from the heat characteristic or from the heating curve. Assuming a linear superposition of the effects of the two gas components, which is the case e.g., at low concentrations in air, the evaluation unit 130 can then calculate the concentrations of both components, e.g., a hydrogen concentration and a water concentration in air, using the heat characteristic and the reference values.

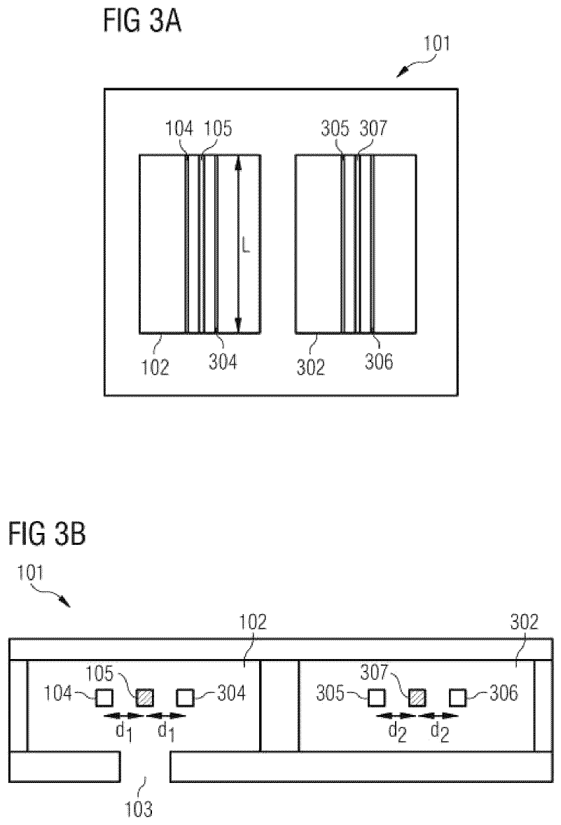

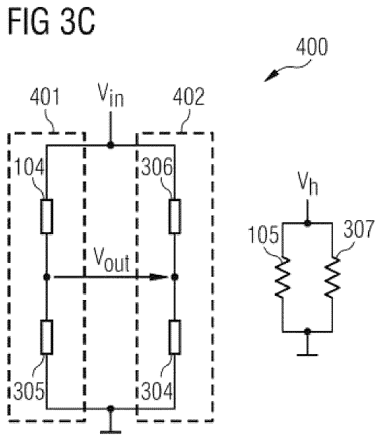

FIG. 3A shows a schematic plan view of a second example implementation of a sensor element 101 for use in a sensor device 100 for measuring a gas concentration. The second implementation comprises a first and a second measuring component 104, 304, a first and a second reference component 305, 306, and a first and a second heating component 105, 307. The measuring and reference components 104, 304, 305, 306 can be formed as bridges made from resistance elements. Likewise, the first and the second heating components 105, 307 can be formed from bridges made from resistance elements. The first measuring component 104, the second measuring component 304 and the first heating component 304 are arranged in the cavity 102 which comprises an opening 103 for receiving a gas to be measured in the cavity 102. The first and the second measuring component 104, 304 are each arranged at a distance from the first heating component 105. For example, the first and the second measuring component 104, 304 are each arranged at an equal distance di from the first heating component 105. Alternatively, the first measuring component 104 can be arranged at a distance from the first heating component 105, which is less or greater than the distance of the second measuring component 304 from the first heating component 105.

The first reference component 305, the second reference component 306 and the second heating component 307 are arranged in a reference cavity 302 which is hermetically sealed and filled with a reference gas. In other words, the sensor element 101 does not comprise a further opening which connects the reference cavity 302 to surroundings of the sensor element 101. The first and the second reference component 305, 306 are each arranged at a distance from the second heating component 307. For example, the first and the second reference component 305, 306 are each arranged at an equal distance d₂ from the second heating component 307. For example, the distance d₂ is equal to the distance d₁. Alternatively, the first reference component 305 can be arranged at a distance from the second heating component 307, which is less or greater than the distance of the second reference component 306 from the second heating component 307. A distance of the first reference component 305 from the second heating element 307 may correspond to the distance of the first measuring component 104 from the first heating element 105. A distance of the second reference component 306 from the second heating element 307 may correspond to the distance of the second measuring component 304 from the first heating element 105.

FIG. 3B shows a schematic cross section of the second example implementation of a sensor element 101. Analogously to FIG. 2B, the sensor element 101 can be a semiconductor component which comprises a housing 200 which is formed from a substrate 201, sidewall elements 202 and a cover 203. The housing 200 thus forms a boundary of the cavity 102 and the reference cavity 302 and defines same. In this example, the substrate 201 comprises the opening 103 to surroundings of the sensor element 101, so that gas for analysis that is to be measured can enter or be introduced into the cavity 102. The opening 103 can alternatively also be arranged in a sidewall element 202 or in the cover 203. The reference cavity 302 is hermetically sealed, that is to say completely surrounded by the housing. The first measuring component 104, the second measuring component 304 and the first heating component 105 are arranged in the cavity 102, so all three components can come into contact with the gas in the cavity 102. The first reference component 305, the second reference component 306 and the second heating component 307 are arranged in the reference cavity 302, so all three components can come into contact with the reference gas, e.g., nitrogen, in the cavity 302. A distance di of the first and second measuring components 104, 304 from the first heating component 105 corresponds in this example implementation to a distance d₂ of the first and second reference components 305, 306 from the second heating component 307.

FIG. 3C shows a circuit diagram of a bridge circuit 400 which may be contained in a sensor element 101 according to the disclosure. The bridge circuit 400 may correspond to a Wheatstone bridge circuit with two measuring components 104, 304 and two reference components 305, 306 (illustrated as resistors in each case) according to the sensor element of FIGS. 3A and 3B. The resistors can be interconnected as illustrated in the circuit diagram, wherein voltage dividers or half bridges 401, 402 of the Wheatstone bridge 400 are formed by one of the measuring components 104, 304 and one of the reference components 305, 306 respectively. The resistance value of the measuring components 104, 304 can change depending on the presence and concentration of a gas for analysis as a function of the heating of the first heating component 105. The resistance value of the reference components 305, 306 can change depending on the presence and concentration of a reference gas as a function of the heating of the second heating component 307. For example, each of the resistors may be similar to the resistance element 104 in FIG. 1. The first heating component 105 and the second heating component 307 can be interconnected parallel to each other, as illustrated in the circuit diagram, so that both components experience the same heating voltage Vₕ. This enables efficient rapid heating at a relatively low heating voltage Vₕ compared to other configurations. For example, both heating components 105, 307 have the same (temperature-dependent) resistance value. Alternatively, the heating components 105, 307 can be interconnected in series.

For measuring the temperature changes, the control circuit arrangement 109 is set up to measure the temperature changes by measuring a bridge voltage Vₒᵤₜ of the Wheatstone bridge 400 as a function of the heating. For this purpose, a measuring voltage Vᵢₙ is applied between a first node and a second node of the bridge circuit 400 by a measuring voltage source of the control circuit arrangement 109, for example a subunit of the voltage supply 110 shown in FIG. 1, wherein the first and the second node are in each case arranged on opposite sides between the half bridges 401, 402. The bridge voltage Vₒᵤₜ of the Wheatstone bridge 400 corresponds to a voltage difference between a third node and a fourth node which are respectively arranged between the measuring and reference component in each of the half bridges 401, 402, as shown in FIG. 3C. The third node can be arranged between the first measuring component 104 and the first reference component 305, while the fourth node can be arranged between the second measuring component 304 and the second reference component 306. The heating voltage Vₕ can be generated by a heating voltage source, for example a subunit of the voltage supply 110 shown in FIG. 1 or a separate voltage supply. An arrangement of measuring and reference components 104, 304, 305, 306 in a Wheatstone bridge 400 and reading out of the bridge voltage Vₒᵤₜ with the measuring voltage Vᵢₙ applied has the effect that offset errors can be compensated and sensitivity is increased.

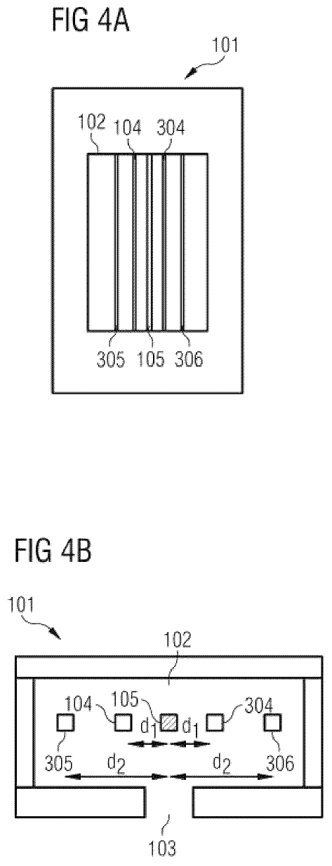

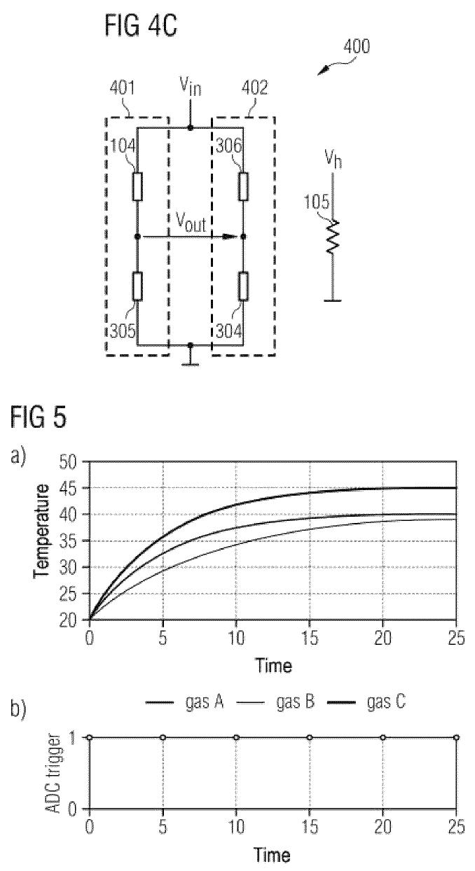

FIG. 4A shows a schematic plan view of a third example implementation of a sensor element 101 for use in a sensor device 100 for measuring a gas concentration. The third implementation comprises a first and a second measuring component 104, 304, a first and a second reference component 305, 306, and a heating component 105, which are all arranged in the cavity 302. The measuring and reference components 104, 304, 305, 306 can be formed as bridges made from resistance elements. Likewise, the heating component 105 can be formed from bridges made from resistance elements. The first and the second measuring component 104, 304 are each arranged at a first distance from the heating component 105. For example, the first and the second measuring component 104, 304 are each arranged at an equal distance di from the heating component 105. Alternatively, the first measuring component 104 can be arranged at a distance from the heating component 105 which is less or greater than the distance of the second measuring component 304 from the heating component 105. The first and the second measuring component 305, 306 are each arranged at a second distance from the heating component 105. For example, the first and the second reference component 305, 306 are each arranged at an equal second distance d₂ from the heating component 105. For example, the distance d₂ is greater than the distance d₁. For example, the second distance d₂ is at least twice as large as the first distance d₁. In this case, the reference components 305, 306 heat up less due to the greater distance from the heating component 105 and are thus less sensitive to the gas in the cavity 102. Such an implementation may be of interest for example for hydrogen sensors for measuring high concentrations. Alternatively, the first reference component 305 can be arranged at a distance from the heating component 105 which is less or greater than the distance of the second reference component 306 from the heating component 105.

FIG. 4B shows a schematic cross section of the third example implementation of a sensor element 101. Analogously to FIGS. 2B and 3B, the sensor element 101 can be a semiconductor component which comprises a housing 200 which is formed from a substrate 201, sidewall elements 202 and a cover 203. The housing 200 thus forms a boundary of the cavity 102 and defines same. In this example, the substrate 201 comprises the opening 103 to surroundings of the sensor element 101, so that gas for analysis that is to be measured can enter or be introduced into the cavity 102. The opening 103 can alternatively also be arranged in a sidewall element 202 or in the cover 203. The first and the second measuring component 104, 304, the first and the second reference component 305, 306 and the heating component 105 are arranged in the cavity 102, so all five components can come into contact with the gas in the cavity 102. A distance d₁ of the first and second measuring components 104, 304 from the heating component 105 corresponds in this example implementation to at least half the distance d₂ of the first and second reference components 305, 306 from the heating component 105.

FIG. 4C shows a circuit diagram of a bridge circuit 400 which may be contained in a sensor element 101 according to FIGS. 4A and 4B. The bridge circuit 400 may correspond to a Wheatstone bridge circuit with two measuring components 104, 304 and two reference components 305, 306 (illustrated as resistors in each case) according to the circuit of FIG. 3C. The measuring principle in this case likewise corresponds to the measuring principle as described with reference to FIG. 3C. As shown in the circuit diagram, the heating component 105 can be supplied with a heating voltage Vₕ from a heating voltage source which may be different from the measuring voltage source.

FIG. 5 is a graph for illustrating the functional principle of the sensor device 100 according to at least one of the example implementations and the typically clear temperature dependence of the measured values during the heating phase. The panel a) of FIG. 5 shows the temperature changes or the fundamental progression of the temperature of the measuring component 104 during a heating phase, e.g., after applying a heating voltage Vₕ to the heating component 105. Here, a measured value of the measuring component 104, for example a resistor or a bridge voltage Vₒᵤₜ, is converted to a temperature of the measuring component 104 in each case. For example, the sensor device comprises a memory in an evaluation unit 130, in which calibration data are stored.

As the temperature profile in panel a) shows, the measuring component 104, which is for example formed by a MEMS wire, is heated owing to the thermal conductivity of the gas for analysis in the cavity 102. The heating constant or heat-up time and the maximum temperature of the measuring component 104 at a given heating voltage Vₕ depend on the gas composition, as illustrated in FIG. 5 for three different gases or gas compositions. For example, gas A is a reference gas, e.g., air. The gas B, e.g., a gas different from air, has the same or at least a very similar thermal conductivity, since the maximum temperature of the measuring component 104 in thermal equilibrium is approximately the same as for gas A, approx. 40° C. here. The time until this maximum temperature is reached is longer for gas B however, since the heat capacity of gas B is higher and thus the temperature profile shows a lower gradient of the heating curve. On the other hand, a third example gas C, which is different from the gases A and B, has a higher thermal conductivity than gas A, which can be seen in the higher maximum temperature of the measuring component 104, approx. 45° C. here. The time constant for reaching this maximum temperature is approximately the same as for gas A however, which is attributable to a similar heat capacity of gases A and C. From this information, it is possible to determine the concentration of a gas component, e.g., H₂ or H₂O in air, provided that the heat capacity and thermal conductivity of the two individual gases in the relevant concentration range are known. For example, the memory of the evaluation unit 130 contains these calibration data. Furthermore, with a gas composition that is generally known and assuming a linear superposition of the effects of the two gas components, which is fulfilled at relatively low concentrations in air, the concentrations of both components can be ascertained.

Panel b) illustrates a trigger signal of the ADC, which causes the measuring voltage Vᵢₙ to be applied selectively and the bridge voltage Vₒᵤₜ to be read out. This illustrates that it is only briefly and at regular time intervals during the heating phase that the measuring voltage can be applied and consequently a measurement can be induced.

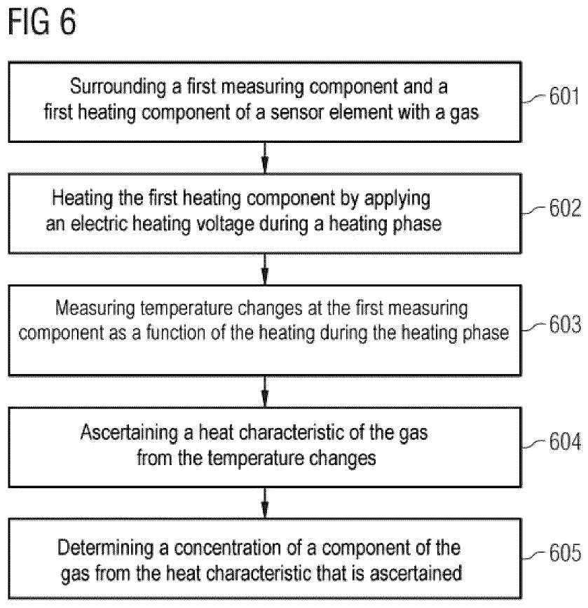

FIG. 6 shows a flowchart of a method for determining a gas concentration using a sensor device 100 according to the disclosure. The method is described in a general form in order to specify aspects of the disclosure qualitatively. The method may contain further aspects.

In step 601, a first measuring component 104 and a first heating component 105 of a sensor element 101 are surrounded by a gas, wherein a concentration of a gas component is to be determined. At 602, a heating phase is initiated by heating up the heating component by applying an electric heating voltage Vₕ. At 603, temperature changes are measured at the first measuring component 104 as a function of the heating during the heating phase. At 604, a heat characteristic of the gas is ascertained from the temperature changes. At 605, a concentration of a component of the gas is determined from the heat characteristic that is ascertained.

It should be pointed out that the description and the drawings only illustrate the principles of the proposed methods and devices. A person skilled in the art will be capable of implementing different arrangements which, although they are not expressly described or shown here, embody the principles of the implementation and are contained within the scope thereof. In addition, all examples and implementations outlined in the present document are intended fundamentally and expressly for explanatory purposes only, in order to help the reader understand the principles of the proposed methods and devices. In addition, all statements in this document which describe principles, aspects and implementations of the implementation and specific examples thereof are also intended to comprise their equivalents.

### ASPECTS

Devices and methods according to the disclosure are explained below based on aspects.

Aspect 1 is a sensor device for measuring a gas concentration, comprising: a sensor element comprising a cavity having an opening for receiving a gas, a first measuring component and a first heating component arranged in the cavity in each case, and a control circuit arrangement which is electrically coupled with the sensor element and set up to control operation of the sensor element. The control circuit arrangement is set up: to heat the first heating component by applying an electric heating voltage during a heating phase, to measure temperature changes at the first measuring component as a function of the heating during the heating phase, to ascertain a heat characteristic of the gas from the temperature changes, and to determine a concentration of a component of the gas from the heat characteristic that is ascertained.

Aspect 2 is a sensor device according to aspect 1, wherein the control circuit arrangement is set up to determine a first concentration of a first component of the gas and a second concentration of a second component of the gas from the heat characteristic that is ascertained.

Aspect 3 is a sensor device according to aspect 1 or 2, wherein the control circuit arrangement for ascertaining the heat characteristic is set up to determine a heating curve from the measured temperature changes, and to ascertain a maximum temperature and/or a gradient of the heating curve.

Aspect 4 is a sensor device according to any one of aspects 1 to 3, wherein the heat characteristic comprises a thermal conductivity and/or a heat capacity of the gas.

Aspect 5 is a sensor device according to any one of aspects 1 to 4, wherein the first heating component and the first measuring component are formed as resistance elements, and the control circuit arrangement for measuring the temperature changes is set up to measure resistance changes of the first measuring component.

Aspect 6 is a sensor device according to any one of aspects 1 to 5, further comprising a substrate, wherein the cavity is formed by a recess in the substrate, and wherein the first heating component is formed by a first bridge and the first measuring component is formed by a second bridge, which bridges are in each case anchored in the substrate and arranged at a distance from each other.

Aspect 7 is a sensor device according to aspect 6, wherein the distance is less than a length of the first and second bridge and is greater than a mean free path length of the gas.

Aspect 8 is a sensor device according to any one of aspects 1 to 7, wherein an impedance of the first measuring component is greater than the impedance of the first heating component.

Aspect 9 is a sensor device according to any one of aspects 1 to 8, wherein the control circuit arrangement for measuring the temperature changes is set up to measure a temperature of the first measuring component during the heating phase by applying an electric measuring voltage at predefined measurement times.

Aspect 10 is a sensor device according to aspect 9, wherein the electric measuring voltage for measuring the temperature is applied during the measurement times and is otherwise switched off.

Aspect 11 is a sensor device according to any one of aspects 1 to 10, wherein the control circuit arrangement for determining the concentration is set up to compare the ascertained heat characteristic with reference values.

Aspect 12 is a sensor device according to any one of aspects 1 to 11, wherein the sensor element further comprises a second measuring component and a first and a second reference component, the first and the second measuring component and the first and the second reference component are interconnected to form a Wheatstone bridge, voltage dividers of the Wheatstone bridge are formed by a measuring component and a reference component respectively, and the control circuit arrangement is further set up to measure the temperature changes by measuring a bridge voltage of the Wheatstone bridge as a function of the heating.

Aspect 13 is a sensor device according to aspect 12, wherein the first and the second measuring component and also the first and the second reference component are formed as bridges made from resistance elements.

Aspect 14 is a sensor device according to aspect 12 or 13, further comprising a second heating component, wherein the second measuring component is arranged in the cavity, the second heating component and the first and the second reference component are arranged in a reference cavity which is hermetically sealed and filled with a reference gas, and the control circuit arrangement is set up to heat the first and the second heating component during the heating phase by applying the electric heating voltage.

Aspect 15 is a sensor device according to aspect 14, wherein the first and the second heating component are arranged in a parallel electrical circuit.

Aspect 16 is a sensor device according to aspect 14 or 15, wherein the first and the second measuring component are each arranged at a first distance from the first heating component, the first and the second reference component are each arranged at a second distance from the second heating component, and the first distance is equal to the second distance.

Aspect 17 is a sensor device according to aspect 12 or 13, wherein the second measuring component and the first and second reference component are arranged in the cavity, the first and the second measuring component are each arranged at a first distance from the first heating component, the first and the second reference component are each arranged at a second distance from the first heating component, and the second distance is greater than the first distance, in particular the second distance is more than twice as large as the first distance.

Aspect 18 is a sensor device according to any one of aspects 1 to 17, wherein the control circuit arrangement is set up to apply the electric heating voltage periodically in pulses.

Aspect 19 is a sensor device according to any one of aspects 1 to 18, wherein the component of the gas is hydrogen.

Aspect 20 is a method for determining a gas concentration, comprising: surrounding a first measuring component and a first heating component of a sensor element with a gas, heating the first heating component by applying an electric heating voltage during a heating phase, measuring temperature changes at the first measuring component as a function of the heating during the heating phase, ascertaining a heat characteristic of the gas from the temperature changes, and determining a concentration of a component of the gas from the heat characteristic that is ascertained.

1. A sensor device for measuring a gas concentration, comprising:
a sensor element comprising a cavity having an opening for receiving a gas, a first measuring component and a first heating component arranged in the cavity in each case; and
a control circuit arrangement which is electrically coupled with the sensor element and set up to control operation of the sensor element, wherein the control circuit arrangement is configured to:
heat the first heating component by applying an electric heating voltage during a heating phase,
measure temperature changes at the first measuring component as a function of heating of the first heating component during the heating phase,
ascertain a heat characteristic of the gas from the temperature changes, and
determine a concentration of a component of the gas from the heat characteristic that is ascertained.

2. The sensor device as claimed in claim 1, wherein the control circuit arrangement is set up to determine a first concentration of a first component of the gas and a second concentration of a second component of the gas from the heat characteristic that is ascertained.

3. The sensor device as claimed in claim 1, wherein the control circuit arrangement for ascertaining the heat characteristic is configured to:
determine a heating curve from the measured temperature changes, and
ascertain a maximum temperature and/or a gradient of the heating curve.

4. The sensor device as claimed in claim 1, wherein the heat characteristic comprises a thermal conductivity and/or a heat capacity of the gas.

5. The sensor device as claimed in claim 1,
wherein the first heating component and the first measuring component are formed as resistance elements, and
wherein the control circuit arrangement for measuring the temperature changes is configured to measure resistance changes of the first measuring component.

6. The sensor device as claimed in claim 1, further comprising a substrate,
wherein the cavity is formed by a recess in the substrate, and
wherein the first heating component is formed by a first bridge and the first measuring component is formed by a second bridge, which bridges are in each case anchored in the substrate and arranged at a distance from each other.

7. The sensor device as claimed in claim 6, wherein the distance is less than a length of the first and second bridge and is greater than a mean free path length of the gas.

8. The sensor device as claimed in claim 1, wherein an impedance of the first measuring component is greater than the impedance of the first heating component.

9. The sensor device as claimed in claim 1, wherein the control circuit arrangement for measuring the temperature changes is configured to measure a temperature of the first measuring component during the heating phase by applying an electric measuring voltage at predefined measurement times.

10. The sensor device as claimed in claim 9, wherein the electric measuring voltage for measuring the temperature is applied during the measurement times and is otherwise switched off.

11. The sensor device as claimed in claim 1, wherein the control circuit arrangement for determining the concentration is configured to compare the heat characteristic with reference values.

12. The sensor device as claimed in claim 1,
wherein the sensor element further comprises a second measuring component and a first and a second reference component,
wherein the first and the second measuring component and the first and the second reference component are interconnected to form a Wheatstone bridge, voltage dividers of the Wheatstone bridge are formed by a measuring component and a reference component, respectively, and
wherein the control circuit arrangement is further configured to measure the temperature changes by measuring a bridge voltage of the Wheatstone bridge as a function of heating of the first heating component.

13. The sensor device as claimed in claim 12, wherein the first and the second measuring component and also the first and the second reference component are formed as bridges made from resistance elements.

14. The sensor device as claimed in claim 12, further comprising a second heating component,
wherein the second measuring component is arranged in the cavity,
wherein the second heating component and the first and the second reference component are arranged in a reference cavity which is hermetically sealed and filled with a reference gas, and
wherein the control circuit arrangement is configured to heat the first and the second heating component during the heating phase by applying the electric heating voltage.

15. The sensor device as claimed in claim 14, wherein the first and the second heating component are arranged in a parallel electrical circuit.

16. The sensor device as claimed in claim 14,
wherein the first and the second measuring component are each arranged at a first distance from the first heating component,
wherein the first and the second reference component are each arranged at a second distance from the second heating component, and
wherein the first distance is equal to the second distance.

17. The sensor device as claimed in claim 12,
wherein the second measuring component and the first and second reference component are arranged in the cavity,
wherein the first and the second measuring component are each arranged at a first distance from the first heating component,
wherein the first and the second reference component are each arranged at a second distance from the first heating component, and
wherein the second distance is greater than the first distance, in particular the second distance is more than twice as large as the first distance.

18. The sensor device as claimed in claim 1, wherein the control circuit arrangement is configured to apply the electric heating voltage periodically in pulses.

19. The sensor device as claimed in claim 1, wherein the component of the gas is hydrogen.

20. A method for determining a gas concentration, comprising:
surrounding a first measuring component and a first heating component of a sensor element with a gas,
heating the first heating component by applying an electric heating voltage during a heating phase,
measuring temperature changes at the first measuring component as a function of the heating during the heating phase,
ascertaining a heat characteristic of the gas from the temperature changes, and
determining a concentration of a component of the gas from the heat characteristic that is ascertained.
